(19) United States
(12) Patent Application Publication
(10) Pub. No.: US 2019/0269614 A1
(43) Pub. Date: Sep. 5, 2019

(54) CHEWABLE TABLET CONTAINING VITAMIN C SODIUM AND PREPARATION METHOD THEREOF

(71) Applicant: GUANGXI SANPOTEL HEALTH INDUSTRY CO. LTD, Nanning (CN)

(72) Inventors: Aiqiang Huang, Nanning (CN); Wen Zhong, Nanning (CN); Aiyi Huang, Nanning (CN)

(21) Appl. No.: 16/396,806

(22) Filed: Apr. 29, 2019

Related U.S. Application Data
(63) Continuation of application No. PCT/CN2017/108406, filed on Oct. 30, 2017.

(30) Foreign Application Priority Data
Oct. 28, 2016 (CN) 201610964478.5

(51) Int. Cl. A61K 9/00; A61K 31/375; A61K 9/20; A23L 33/15; A23L 27/30; A23L 29/30; A23L 29/219; A23L 29/262; A23L 29/00

(52) U.S. Cl. CPC A61K 9/0056; A23V 2002/00; A61K 9/2059; A61K 9/2095; A61K 9/2018; A61K 9/2054; A61K 9/2027; A61K 9/2013; A23L 33/15; A23L 27/32; A23L 27/33; A23L 29/37; A23L 29/219; A23L 29/262; A23L 29/04; A23L 29/055; A61K 31/375

(57) ABSTRACT

The present application provides a chewable tablet containing vitamin C sodium. The chewable tablet includes the following components in parts by weight: 20-100 parts of vitamin C sodium, 100-500 parts of starch, 10-30 parts of sweetener, 5-20 parts of mannitol, 30-100 parts of microcrystalline cellulose, 5-20 parts of povidone K-30, 20-60 parts of sodium carboxymethyl starch, 5-20 parts of sodium dodecyl sulfate, 1-5 parts of magnesium stearate and 0.1-0.5 part of mint essence. The present application is easy to prepare, sweet in taste and stable in quality.

### CROSS REFERENCE TO RELATED APPLICATIONS

The present application is a Continuation Application of PCT Application No. PCT/CN2017/108406 filed on Oct. 30, 2017, which claims the priority of Chinese Patent Application No. 201610964478.5 filed on Oct. 28, 2016. The contents of all of the above are hereby incorporated by reference.

### BACKGROUND OF THE INVENTION

### Field of the Invention

The present application belongs to the fields of medicines, health foods and foods, and relates to a composition preparation and a preparation method thereof, in particular, to a chewable tablet containing vitamin C sodium and a preparation method thereof.

### Related Arts

Vitamin C is a basic and common clinical drug or nutritional supplement and one of antioxidant vitamins. Vitamin C participates in the hydroxylation reaction in vivo, is necessary for the formation of intercellular adhesions of bones, teeth, connective tissues and non-epithelial tissues, and can maintain normal functions of teeth, bones and blood vessels and increase the resistance to diseases. Vitamin C is one of the essential nutritional elements for human body and is widely used in the prevention and treatment of various diseases.

Tablets are stable in quality and convenient to take, and are the most basic and common preparation form. However, for children, the elderly and patients with dysphagia, ordinary tablets are often difficult to take. Long-term medication may even cause psychological resistance to drugs. Chewable tablets can make up for this deficiency. Chewable tablets are tablets which can be chewed and then swallowed. The sizes of chewable tablets is generally the same as that of ordinary tablets. Chewable tablets can be made into special-shaped tablets in different shapes as needed. After being chewed, the tablets are easy to swallow, the surface area of the tablets is increased, and the dissolution and absorption of the drugs in the body can be promoted. For drugs difficult to disintegrate, disintegration can be accelerated and the drug efficacy can be improved when they are made into chewable tablets. Chewable tablets are convenient to take, can be taken on time even without water, are especially suitable for children, the elderly, patients with dysphagia or poor gastrointestinal function, and can reduce the burden of drugs on gastrointestinal tract. Therefore, chewable tablets are more and more popular.

As vitamin C is one of the important nutrients necessary to maintain the normal physiological function of human body, vitamin C chewable tablets have been favored by people in recent years. However, due to a problem that vitamin C is prone to oxidation and loss of efficacy, the use effect and shelf life of vitamin C chewable tablets are greatly affected. Furthermore, vitamin C chewable tablets have strong acidity after dissolution, and will cause great irritation to oral cavity, throat, esophagus and gastric mucosa, so the vitamin C chewable tablets are not suitable for being taken for a long time, nor are the vitamin C chewable tablets suitable for being taken with acidic drugs at the same time. There are certain restrictions in daily use as a nutritional agent or for the treatment or prevention of clinical diseases.

Vitamin C sodium is the sodium salt of vitamin C, the pH value of the aqueous solution thereof is nearly neutral, and its function is the same as that of vitamin C. However, as a sodium salt, its performance is more stable; besides, vitamin C sodium no longer has the strong acidity of vitamin C, thus being able to be taken together with various drugs for a long term, so vitamin C sodium is better than vitamin C. Vitamin C sodium is a widely used vitamin C enhancer at home and abroad, and has gradually replaced vitamin C. In the present application, vitamin C sodium is directly prepared into chewable tablets, and clinical trials show that the preparation is safe and effective, easy to prepare, sweet and palatable, and stable in quality.

### SUMMARY OF THE INVENTION

In view of problems that existing vitamin C chewable tablets have strong acidity after dissolution, and will cause great irritation to oral cavity, throat, esophagus and gastric mucosa, so they are not suitable for being taken for a long time, and vitamin C is prone to oxidation and loss of efficacy during production and storage, etc., the present application provides a chewable tablet containing vitamin C sodium and a preparation method thereof. The present application solves problems that vitamin C is unstable in quality during clinical application and not suitable for being taken for a long time. The chewable tablet containing vitamin C sodium is sweet and palatable, has high bioavailability, is easy to prepare and stable in quality, and can be used for treating scurvy, infectious diseases, purpura with muscle weakness, paralysis, arrhythmia or renal dysfunction in the long term, or for preventing and assisting in treating malignant tumors, cardiovascular and cerebrovascular diseases, infectious diseases or autoimmune diseases and the like, or for long-term use as a nutritional supplement.

In order to solve the above technical problems, the present application are realized through the following technical solution:

A chewable tablet containing vitamin C sodium includes the following components in parts by weight: 20-100 parts of vitamin C sodium, 200-500 parts of starch, 5-20 parts of mannitol, 30-100 parts of microcrystalline cellulose, 5-20 parts of povidone K-30, 10-60 parts of sodium carboxymethyl starch, 5-20 parts of sodium dodecyl sulfate, 1-5 parts of magnesium stearate, 10-30 parts of sweetener and 0.1-0.5 part of mint essence.

The chewable tablet containing vitamin C sodium preferably includes the following components in parts by weight: 30 parts of vitamin C sodium, 361.75 parts of starch, 10 parts of mannitol, 60 parts of microcrystalline cellulose, 10 parts of povidone K-30, 40 parts of sodium carboxymethyl starch, 10 parts of sodium dodecyl sulfate, 3 parts of magnesium stearate, 20 parts of sweetener and 0.25 part of mint essence.

According to the chewable tablet containing vitamin C sodium, the sweetener is preferably selected from the group consisting of aspartame, neotame, and mogroside.

A preparation method of the chewable tablet containing vitamin C sodium includes the following steps:

S1: respectively taking vitamin C sodium, starch, mannitol, microcrystalline cellulose, povidone K-30, sodium carboxymethyl starch, sodium dodecyl sulfate and sweetener, pulverizing, and sieving with a 100-mesh sieve for later use;

S2: respectively weighing the raw and auxiliary materials in step S1 according to the formula amount, and uniformly mixing by an equal increment method to obtain mixed powder;

S3: taking the mixed powder in step S2, adding 70-80% ethanol solution while stirring to prepare a damp mass, granulating and sieving, drying for 4 hours, controlling the moisture content to be 2-4%, and sizing to obtain granules; and

S4: weighing magnesium stearate and mint essence according to the formula amount, evenly mixing with the granules in step S3, and compressing the granules into tablets.

Further, the drying temperature in step S3 is 60-65° C., so that the obtained granules have moderate hardness so as to be more favorable for tabletting. A screen used for sieving is preferably a 14-30 mesh screen.

The present application has the beneficial effects:

1. The problems that existing vitamin C chewable tablets have strong acidity after dissolution, and will cause great irritation to oral cavity, throat, esophagus and gastric mucosa, so they are not suitable for being taken for a long time, and vitamin C is prone to oxidation and loss of efficacy during production and storage are solved, and the product has better stability and more exact curative effect, is suitable for being taken for a long time by consumers or patients, and has no side effects;

2. The present application can be used for treating diseases such as scurvy, infectious diseases, purpura with muscle weakness, paralysis, arrhythmia or renal dysfunction, or for preventing and assisting in treating severe difficult and complicated diseases such as malignant tumors, cardiovascular and cerebrovascular diseases, infectious diseases, autoimmune diseases and other major difficult diseases, or for being taken for a long time as a nutritional supplement; and

3. The chewable tablet containing vitamin C sodium of the present application is easy to prepare, good in granule fluidity, non-sticking during tabletting, smooth in surface, uniform in color, moderate in hardness, sweet and palatable, stable in quality and convenient to carry and eat, is especially suitable for children and the elderly, has high bioavailability, and is safer and more effective to use.

### DETAILED DESCRIPTION OF THE INVENTION

Although the specification concludes with claims particularly pointing out and distinctly claiming the protection of the present application, it is believed that the following description will facilitate a better understanding of the present application.

As used herein, the word “preferred” and variants thereof refer to embodiments of the present application which are capable of providing specific benefits under specific circumstances. However, other embodiments may also be preferred under the same or other circumstances. Furthermore, the detailed description of one or more preferred embodiments does not indicate that other embodiments are useless and is not intended to exclude other embodiments from the scope of the present application.

I. Selection of Preparation Conditions

1. Selection of Wetting Agent

A wetting agent can wet powder particles to obtain viscosity so as to facilitate the preparation of the granules and compress the granules into tablets. In the present application, dry tabletting and wet granulation and tabletting are compared, and since dry tabletting has a high requirement for equipment and cannot generate a good effect by adopting a common tabletting machine, wet granulation and tabletting is adopted herein. In the experiment, 20% maltodextrin, 30% starch slurry, 40% sucrose syrup, 5% hydroxypropyl methylcellulose aqueous solution and 80% ethanol were compared as the wetting agent. Results are shown in Table 1.

TABLE 1
Investigation Results of Wetting Agent

| Serial | | | Granule |
|---|---|---|---|
| number | Adhesive | Granulation condition | appearance |
| 1 | 20% maltodextrin | slightly clustering, | relatively |
| | | agglomerating easily | hard |
| | | during granulation | granules |
| 2 | 30% starch slurry | clustering, agglomerating | hard granules |
| | | easily during granulation | |
| 3 | 40% sucrose syrup | clustering, agglomerating | hard granules |
| | | easily during granulation | |
| 4 | 5% hydroxypropyl | clustering, agglomerating | hard granules |
| | methylcellulose | easily during granulation | |
| | aqueous solution | | |
| 5 | 80% ethanol | clustering, not | appropriate |
| | | agglomerating during | granule |
| | | granulation | tightness |

From the test results in Table 1, it can be seen that 80% ethanol used in the present application has the best granulation effect, so in the present application, ethanol is selected as the wetting agent. The present application continues to optimize the concentration of ethanol, and the test results are shown in Table 2.

TABLE 2
Investigation Results of Ethanol Concentration

| Serial | | | Granule |
|---|---|---|---|
| number | Wetting agent | Granulation condition | appearance |
| 1 | 50% ethanol | clustering, hard to granulate | tight granules |
| 2 | 60% ethanol | clustering, hard to granulate | tight granules |
| 3 | 70% ethanol | clustering, not agglomerating | appropriate |
| | | during granulation | granule |
| | | | tightness |
| 4 | 80% ethanol | clustering, not agglomerating | appropriate |
| | | during granulation | granule |
| | | | tightness |
| 5 | 90% ethanol | not clustering, hard to | loose granules |
| | | granulate | |

From the test results in Table 2, it can be seen that the 70%-80% ethanol used in the present application has a good granulation effect, tightness is too high for a lower concentration but too low for a higher concentration, which both affect granulation, so in the present application, the 70%-80% ethanol is selected as the wetting agent.

2. Lubricant Selection

During tabletting, in order to increase granule fluidity, and ensure good filling and even distribution of tablet density, a certain amount of lubricant needs to be added to solve the problems of poor granule fluidity and sticking during tabletting. In the experiment of the present application, tabletting conditions when magnesium stearate, micro-powder silica gel, talcum powder, sodium dodecyl sulfate and mannitol were used as the lubricant were compared, and results are shown in Table 3.

TABLE 3
Lubricant Trial Results

| | | Tabletting | Tablet weight | | | | |
|---|---|---|---|---|---|---|---|
| Lubricant | Fluidity | condition | difference | Appearance | Hardness | Friability | taste |
| mannitol | good | non-sticking | meeting | intact and smooth | 54 N | 0.39% | crisp, |
| | fluidity | | specification | tablet surface, | | | not |
| | | | | uniform color | | | gritty |
| micro-powder | good | non-sticking | meeting | tablet surface not | 29 N | 1.21% | too |
| silica gel | fluidity | | specification | intact and smooth | | | crisp, |
| | | | | enough, | | | not |
| | | | | nonuniform color | | | gritty |
| talcum | poor | sticking | meeting | tablet surface not | 98 N | 0.12% | hard |
| powder | fluidity | | specification | intact and smooth | | | and |
| | | | | enough, | | | gritty |
| | | | | nonuniform color | | | |
| sodium | good | non-sticking | meeting | intact and smooth | 59 N | 0.42% | crisp, |
| dodecyl | fluidity | | specification | tablet surface, | | | not |
| sulfate | | | | uniform color | | | gritty |
| magnesium | good | non-sticking | meeting | intact and smooth | 49 N | 0.34% | crisp, |
| stearate | fluidity | | specification | tablet surface, | | | not |
| | | | | uniform color | | | gritty |

From the test results in Table 3, it can be seen that when magnesium stearate, sodium dodecyl sulfate and mannitol are used, fluidity is good and sticking during tabletting is avoided, and prepared tablets have appropriate hardness and smooth and beautiful surfaces; when micro-powder silica gel is added, fluidity is good and sticking during tabletting is avoided, but the hardness of prepared tablets is low; and when talcum powder is added, fluidity is poor and sticking occurs during tabletting, and prepared tablets have large hardness. Therefore, magnesium stearate, sodium dodecyl sulfate and mannitol are selected as the lubricant in the present application. In order to adjust the taste of the chewable tablets and in consideration of production costs, in the experiment of the present application, the proportion of magnesium stearate to sodium dodecyl sulfate to mannitol was also compared, and the results show that the taste is the best when the proportion of magnesium stearate to sodium dodecyl sulfate to mannitol is 3:3:1 or 4:4:1.

4. Selection of Flavoring Agent

Only chewable tablets with good taste can be easily accepted by people, so flavor and taste are very important, and this is often realized through the selection of flavoring agents such as sweetener and essence. In the experiment of the present application, the following sweeteners were compared: sucrose, lactose, glucose, stevioside, disodium glycyrrhizinate, aspartame, saccharin sodium, sodium cyclamate, mogroside, neotame and aclame. The results show that when aspartame, mogroside and neotame were used in combination with mint essence within the range of “10-30 parts of sweetener”, taste and flavor were good, sweetness was moderate and no discomfort was caused, where aspartame produced the best taste. Therefore, aspartame, mogroside and neotame are selected as sweeteners in the present application, and the addition amount thereof is specified to be 10-30 parts.

II. Preparation Method of Chewable Tablet Containing Vitamin C Sodium

### Embodiment 1

A preparation method of the chewable tablet containing vitamin C sodium includes the following steps:

S1: respectively taking vitamin C sodium, starch, mannitol, microcrystalline cellulose, povidone K-30, sodium carboxymethyl starch, sodium dodecyl sulfate and neotame, pulverizing, and sieving with a 100-mesh sieve for later use;

S2: respectively weighing the following raw and auxiliary materials in step S1 in parts by weight: 30 kg of vitamin C sodium, 361.75 kg of starch, 10 kg of mannitol, 60 kg of microcrystalline cellulose, 10 kg of povidone K-30 kg, 40 kg of sodium carboxymethyl starch, 10 kg of sodium dodecyl sulfate and 20 kg of neotame, and uniformly mixing by an equal increment method to obtain mixed powder;

S3: taking the mixed powder in step S2, adding 80% ethanol solution while stirring to prepare a damp mass, sieving with a 20-mesh sieve and granulating, drying for 4 hours at 62° C., controlling the moisture content to be 2-4%, and sieving with a 20-mesh sieve and sizing to obtain granules; and

S4: weighing the following components in parts by weight: 3 kg of magnesium stearate and 0.25 kg of mint essence, evenly mixing with the granules in step S3, and compressing the granules into tablets.

### Embodiment 2

A preparation method of the chewable tablet containing vitamin C sodium includes the following steps:

S1: respectively taking vitamin C sodium, starch, mannitol, microcrystalline cellulose, povidone K-30, sodium carboxymethyl starch, sodium dodecyl sulfate and aspartame, pulverizing, and sieving with a 100-mesh sieve for later use;

S2: respectively weighing the following raw and auxiliary materials in step S1 in parts by weight: 20 kg of vitamin C sodium, 200 kg of starch, 5 kg of mannitol, 30 kg of microcrystalline cellulose, 5 kg of povidone K-30 kg, 10 kg of sodium carboxymethyl starch, 5 kg of sodium dodecyl sulfate and 10 kg of aspartame, and uniformly mixing by an equal increment method to obtain mixed powder;

S3: taking the mixed powder in step S2, adding 70% ethanol solution while stirring to prepare a damp mass, sieving with a 30-mesh sieve and granulating, drying for 4 hours at 60° C., controlling the moisture content to be 2-4%, and sieving with a 30-mesh sieve and sizing to obtain granules; and

S4: weighing the following components in parts by weight: 1 kg of magnesium stearate and 0.1 kg of mint essence, evenly mixing with the granules in step S3, and compressing the granules into tablets.

### Embodiment 3

A preparation method of the chewable tablet containing vitamin C sodium includes the following steps:

S1: respectively taking vitamin C sodium, starch, mannitol, microcrystalline cellulose, povidone K-30, sodium carboxymethyl starch, sodium dodecyl sulfate and mogroside, pulverizing, and sieving with a 100-mesh sieve for later use;

S2: respectively weighing the following raw and auxiliary materials in step S1 in parts by weight: 100 kg of vitamin C sodium, 500 kg of starch, 20 kg of mannitol, 100 kg of microcrystalline cellulose, 20 kg of povidone K-30, 60 kg of sodium carboxymethyl starch, 20 kg of sodium dodecyl sulfate and 30 kg of mogroside, and uniformly mixing by an equal increment method to obtain mixed powder;

S3: taking the mixed powder in step S2, adding 75% ethanol solution while stirring to prepare a damp mass, sieving with a 14-mesh sieve and granulating, drying for 4 hours at 65° C., controlling the moisture content to be 2-4%, and sieving with a 14-mesh sieve and sizing to obtain granules; and

S4: weighing the following components in parts by weight: 5 kg of magnesium stearate and 0.5 kg of mint essence, evenly mixing with the granules in step S3, and compressing the granules into tablets.

III. Stability Test

Samples from Embodiments 1-3 were packaged with double-layer aluminum-plastic composite films respectively, and placed in a stability acceleration test chamber for a three-month acceleration test, where the temperature is 40±2° C., and the relative humidity is 75±5%. Results show that compared with the test results of the samples at time 0, the samples from Embodiments 1-3 have no obvious changes in terms of key stability indexes such as appearance, taste, hardness, friability and main component content, and taste and flavor are not changed either, indicating that the samples from Embodiments 1-3 of the present application are stable in quality and can meet stability requirements for storage, transportation, and use. The test results are shown in Table 4.

TABLE 4
Stability Test Results of Chewable Tablet Containing Vitamin C Sodium

| | | | | | | Vitamin C |
|---|---|---|---|---|---|---|
| | | | | | | sodium content |
| | | | | | | (by marked |
| Embodiment | Time | Appearance | Taste | Hardness | Friability | amount %) |
| Embodiment 1 | month | intact and | sweet and | 49 N | 0.32% | 99.75 |
| | 0 | smooth tablet | palatable, crisp, | | | |
| | | surface, | not gritty | | | |
| | | uniform color | | | | |
| | month | intact and | sweet and | 51 N | 0.35% | 99.82 |
| | 1 | smooth tablet | palatable, crisp, | | | |
| | | surface, | not gritty | | | |
| | | uniform color | | | | |
| | month | intact and | sweet and | 52 N | 0.39% | 99.94 |
| | 2 | smooth tablet | palatable, crisp, | | | |
| | | surface, | not gritty | | | |
| | | uniform color | | | | |
| | month | intact and | sweet and | 52 N | 0.39% | 99.61 |
| | 3 | smooth tablet | palatable, crisp, | | | |
| | | surface, | not gritty | | | |
| | | uniform color | | | | |
| Embodiment 2 | month | intact and | sweet and | 53 N | 0.39% | 99.12 |
| | 0 | smooth tablet | palatable, crisp, | | | |
| | | surface, | not gritty | | | |
| | | uniform color | | | | |
| | month | intact and | sweet and | 51 N | 0.37% | 99.04 |
| | 1 | smooth tablet | palatable, crisp, | | | |
| | | surface, | not gritty | | | |
| | | uniform color | | | | |
| | month | intact and | sweet and | 54 N | 0.41% | 98.96 |
| | 2 | smooth tablet | palatable, crisp, | | | |
| | | surface, | not gritty | | | |
| | | uniform color | | | | |
| | month | intact and | sweet and | 54 N | 0.41% | 99.07 |
| | 3 | smooth tablet | palatable, crisp, | | | |
| | | surface, | not gritty | | | |
| | | uniform color | | | | |
| Embodiment 3 | month | intact and | sweet and | 43 N | 0.31% | 100.04 |
| | 0 | smooth tablet | palatable, crisp, | | | |
| | | surface, | not gritty | | | |
| | | uniform color | | | | |
| | month | intact and | sweet and | 42 N | 0.29% | 100.12 |
| | 1 | smooth tablet | palatable, crisp, | | | |
| | | surface, | not gritty | | | |
| | | uniform color | | | | |
| | month | intact and | sweet and | 44 N | 0.33% | 99.88 |
| | 2 | smooth tablet | palatable, crisp, | | | |
| | | surface, | not gritty | | | |
| | | uniform color | | | | |
| | month | intact and | sweet and | 44 N | 0.34% | 99.79 |
| | 3 | smooth tablet | palatable, crisp, | | | |
| | | surface, | not gritty | | | |
| | | uniform color | | | | |

What is claimed is:

1. A chewable tablet containing vitamin C sodium, comprising the following components in parts by weight: 20-100 parts of vitamin C sodium, 200-500 parts of starch, 5-20 parts of mannitol, 30-100 parts of microcrystalline cellulose, 5-20 parts of povidone K-30, 10-60 parts of sodium carboxymethyl starch, 5-20 parts of sodium dodecyl sulfate, 1-5 parts of magnesium stearate, 10-30 parts of sweetener and 0.1-0.5 part of mint essence.

2. The chewable tablet containing vitamin C sodium according to claim 1, comprising the following components in parts by weight: 30 parts of vitamin C sodium, 361.75 parts of starch, 10 parts of mannitol, 60 parts of microcrystalline cellulose, 10 parts of povidone K-30, 40 parts of sodium carboxymethyl starch, 10 parts of sodium dodecyl sulfate, 3 parts of magnesium stearate, 20 parts of sweetener and 0.25 part of mint essence.

3. The chewable tablet containing vitamin C sodium according to claim 1, wherein the sweetener is selected from the group consisting of aspartame, neotame, and mogroside.

4. A preparation method of the chewable tablet containing vitamin C sodium according to claim 1, comprising the following steps:
S1: respectively taking vitamin C sodium, starch, mannitol, microcrystalline cellulose, povidone K-30, sodium carboxymethyl starch, sodium dodecyl sulfate and sweetener, pulverizing, and sieving with a 100-mesh sieve for later use;
S2: respectively weighing the raw and auxiliary materials in step S1 according to the formula amount, and uniformly mixing by an equal increment method to obtain mixed powder;
S3: taking the mixed powder in step S2, adding 70-80% ethanol solution while stirring to prepare a damp mass, granulating and sieving, drying for 4 hours, controlling the moisture content to be 2-4%, and sizing to obtain granules; and
S4: weighing magnesium stearate and mint essence according to the formula amount, evenly mixing with the granules in step S3, and compressing the granules into tablets.

5. The preparation method of the chewable tablet containing vitamin C sodium according to claim 4, wherein the drying temperature in step S3 is 60-65° C.

6. The preparation method of the chewable tablet containing vitamin C sodium according to claim 4, wherein a screen used for sieving in step S3 is a 14-30 mesh screen.
